(19) United States
(12) Patent Application Publication
(10) Pub. No.: US 2005/0267510 A1
(43) Pub. Date: Dec. 1, 2005

(54) Device for the endovascular treatment of intracranial aneurysms

(76) Inventor: Nasser Razack, Ann Arbor, MI (US)

Correspondence Address: HARNESS, DICKEY & PIERCE, P.L.C., P.O. BOX 828, BLOOMFIELD HILLS, MI, 48303, US

(21) Appl. No.: 10/854,293

(22) Filed: May 26, 2004

(51) Int. Cl. A61M 29/00

(52) U.S. Cl. 606/200

(57) ABSTRACT

A method and device for intravascular treatment of various vascular and intravascular issues, such as aneurysms, is disclosed. The device generally includes a multiple member, bead strand or device that includes a plurality of members interconnected with one another in a substantially flexible manner. The device may be positioned through a catheter or micro-catheter substantially intravascularly to occlude or embolize an aneurysm to substantially eliminate the possibility of perforation or rupture of the aneurysm.

### FIELD

The present invention relates generally to endovascular procedures, and particularly to methods and apparatus to form an embolism of a vascular site.

### BACKGROUND

As is known in the art, it may often be desirable to disrupt blood flow or supply to a selected area. Generally, in an anatomy, various vessels and veins carry blood from various portions of the anatomy to other portions of the anatomy. The blood is delivered through the vessels and veins that have a substantially constant cross-section. Nevertheless, for various reasons, such as injuries, disease, cancer, genetic predisposition, and the like, it is desirable to stop blood flow to a selected area. For example, an aneurysm may cause injury to an individual if the aneurysm perforates or ruptures, thus reducing blood flow to the parent vessel from which the aneurysm arises or the resultant hemorrhage causing injury, i.e. subarachnoid hemorrhage.

Although it is known in the art to attempt to fill the aneurysm with a selected material, generally the procedures require much care, skill and finesse to provide the material and structure to the aneurysm. For example, a balloon may be provided that may be filled with a selected material, such as a polymerizing material, to substantially fill an aneurysm. Nevertheless, it may be difficult to guide the balloon through a selected vessel and through a catheter to a selected position. Furthermore, positioning the balloon in a selected position may also be difficult.

Various other techniques include providing micro coils or coils through a catheter to an aneurysm to fill the aneurysm with the coils. The coils may be augmented with a bioabsorbable polymer (such as polyglycolic acid or polylactic acid) or hydrogel that may incite an inflammatory reaction (bioabsorbable polymer) or expand (hydrogel) after a selected period of time. These modified coils have an advantage over “bare” platinum coils in that by inciting an inflammatory reaction (bioabsorbable polymer) or expanding (hydrogel), they are able to embolize a significantly greater portion of the aneurysm lumen. Filling more of the aneurysm lumen improves the durability of the endovascular treatment and reduces the rate of recannalization of the aneurysm. Nevertheless, the coils are often simply coated with a hydrogel material which allows for only limited expansion. One concern is that the bioabsorbable polymer and hydrogel coated coils tend to be “stiffer” or have a higher rigidity than bare platinum coils. Since the wall of an aneurysm is far weaker than a normal arterial wall, it is not beneficial to increase the rigidity of the endovascular device. The more rigid an endovascular coil is, the greater the chance of it perforating an aneurysm during deployment.

Therefore, it is desirable to provide a system and method to embolize an aneurysm, or other appropriate system, with a substantially flexible apparatus that allows for easy positioning of the apparatus with the potential to significantly increase the degree of embolic packing of an aneurysm compared to a bare platinum coil without increasing the intrinsic rigidity of the embolic device or decreasing its rigidity compared to a bare platinum coil. Therefore, it is desirable to provide a material that may be easily positioned within the aneurysm and provide a substantially large fill factor or percentage to substantially embolize the aneurysm in a directed procedure. In short, it is desirable to provide a device which retains the softness of bare platinum coils while being coated or impregnated with a therapeutic material, which can include, but is not limited to, any one or combination of hydrogel, bioabsorbable polymer (such as polyglycolic acid or polylactic acid), antibodies or other medically therapeutic substances.

### SUMMARY

A method and apparatus that may provide for embolizing an aneurysm or an emolization system. Generally, the apparatus includes a bead that may be formed in any appropriate shape or size, such as substantially spherical or a round member. The bead or member may be interconnected with a plurality of similar members to allow for positioning of a selected number of members in an aneurysm. The spherical members may be substantially solid, hollow, porous, cage members or other appropriate shapes or geometry. Nevertheless, the members may be interconnected with a substantially rigid or flexible member over a substantially short distance to allow for a flexibility of the apparatus for easy positioning of the apparatus within the aneurysm. The apparatus may include a selected hydrogel, either coated on a substantial solid sphere, or included within a substantially porous sphere, to allow for a maximum expansion of the hydrogel and a maximum inclusion of a volume of the hydrogel. The apparatus may be included with a plurality of other systems, such as balloons, coils, and the like to allow for a substantially complete and selected embolization of an aneurysm.

According to various embodiments, an embolization system may include a flexible conduit substantially interconnecting one or a plurality of spherical functional units positioned at spaced intervals along the flexible conduit. A hydrogel coating may be carried on or hydrogel may be contained within each of the plurality of spherical functional units.

According to various embodiments an embolization system may include a plurality of spherical functional units wherein at least one of the plurality of spherical functional units defines an internal cavity. A flexible conduit fixedly supporting the plurality of spherical functional units, the flexible conduit may extend though the internal cavity of each of the spherical units. Also, a hydrogel solution may be disposed within the internal cavity of each of the plurality of spherical functional units.

According to various embodiments an embolization system for substantially obstructing an aneurysm may include a first member positioned relative to the aneurysm and a second member positioned relative to the aneurysm. A connection member may interconnect the first member and the second member. The first member and the second member may be moveable relative to one another via the connection member for implantation of the first member and the second member.

According to various embodiments a method of providing an embolization system to a vascular region may include an expanding element. The method may include providing a package including a volume and placing an expanding element in the volume. A bore may also be provided in the package such that the expanding element may interact with an environment surrounding the package. Also the package may be guided to the vascular region.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1A is a portion of an embolization system according to a selected embodiment;

FIG. 1B is a portion of an embolization system according to a selected embodiment;

FIG. 2 is a segment of an embolization system according to various embodiments;

FIG. 3 is a portion of an embolization system according to a selected embodiment;

FIG. 4 is a portion of an embolization system according to a selected embodiment;

FIG. 5 is a portion of an embolization system according to a selected embodiment;

FIG. 6 is a portion of an embolization system according to a selected embodiment;

FIG. 7 is a portion of an embolization system according to a selected embodiment;

FIG. 8 is a portion of an embolization system according to a selected embodiment;

FIG. 9 is a diagrammatic view of an embolization system according to various embodiments;

FIG. 10 is an environmental diagrammatic view of a positioning of an embolization system according to various embodiments; and

FIG. 11 is an environmental diagrammatic view of a positioning of an embolization system according to various embodiments.

### DETAILED DESCRIPTION OF VARIOUS EMBODIMENTS

The following description of the preferred embodiments is merely exemplary in nature and is in no way intended to limit the invention, its application, or uses. Moreover, various combinations may be included in the various embodiments.

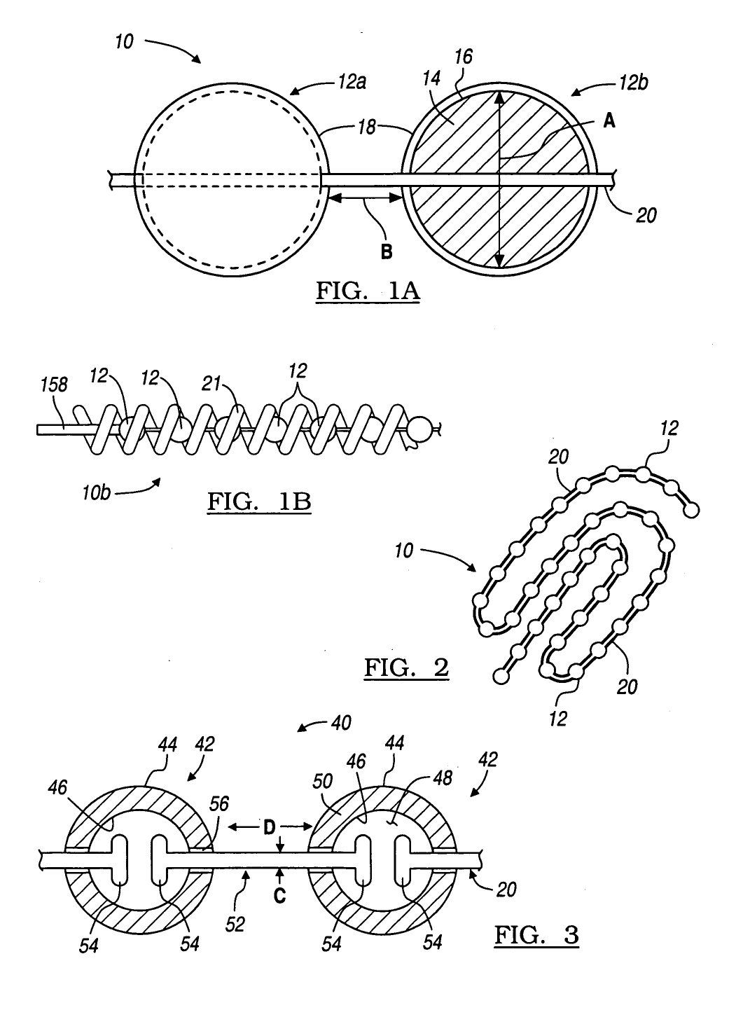

With reference to FIG. 1A, a vascular occluding device 10 or emolization system, which may also be referred to as an embolizing device, is illustrated. The vascular occluding device 10 generally includes a bead 12 or a plurality of the beads 12. With particular reference to FIG. 1A, first bead 12a is illustrated substantially whole while second bead 12b is illustrated in cross-section. It will be understood that the occluding device 10 may have any appropriate number of beads 12 or other occluding portions, such as those discussed herein but not limited thereto.

The beads 12 may include a substantially solid core 14 that extends or defines a surface 16. Generally, the beads 12 may include a surface or core diameter A of about 0.01 inches to about 0.02 inches (about 0.25 mm to about 0.51 mm). It will be understood, however, that the diameter of the beads 12 may be any appropriate diameter, as discussed herein. Generally, the diameter A of the beads 12 will be provided at an appropriate diameter to allow for a successful occlusion of a vascular region or embolization of an aneurysm. Although the beads 12 may be allowed to pass through a selected cannula diameter, such as a micro cannula for passing through a vascular region, it will be understood that the beads 12 may be of any appropriate shape or size. The beads 12 need not be spherical.

The beads 12 may be formed of any appropriate material. For example, the beads 12 may be formed of a substantially radio-opaque material. The various appropriate radio-opaque materials include, but are not limited to, tantalum, stainless steel, gold, platinum, titanium, tungsten, and other appropriate metals or metal alloys. In addition, the beads 12, particularly the core 14, may be formed of a polymer material that is appropriate for implantation into an anatomy. The polymer material may also include a radio-opaque material, such as microcrystalline beads, a powder, or other appropriate materials. Therefore, the beads 12 may be substantially viewable using various imaging devices, such as a X-ray, a fluoroscope device, or other appropriate imaging devices. The beads 12, generally spaced along a strand 20, may provide for ease of viewing the entire occluding device 10. The imaging device is generally able to image the beads 12 including a substantially radio-opaque material after implantation of the beads 12 into an anatomy.

In addition, the beads 12 may be interconnected with any appropriate portion, either at an end of the occluding device 10 or substantially intermittent therein. For example, the beads 12 may be interconnected with various portions, such as metallic portions including those that may be formed from, but not limited to platinum and gold, or may be interconnected with various shape memory metals or polymers. A shape memory polymer may have a glass transition temperature of below about 25° C. for forming a selected structure. In addition, the beads 12 may be interconnected with a selected hydrogel, amorphous gel, polymer, suture, polymer suture or fiber, according to various embodiments including those discussed herein. In addition, the coil, or any appropriate structure may be substantially interwoven with the beads 12, if the bead includes a portion that allows for a strand to be interwoven therewith. As discussed herein, and according to various embodiments, a bead may include a structure that is substantially porous or woven. Therefore, a bead may provide a position that allows for a strand to be interwoven therewith to extend externally from the bead in any appropriate configuration.

The beads 12 may also include a coating 18. Generally the coating 18 is positioned around the surface 16 of the core 14. The coating 18 may be any appropriate thickness, but generally may be limited to maintain a thickness to allow the passing of the beads 12 through a selected cannula. The coating 18 may include a substantially minimal thickness to provide for a selected activity or functionality. The coating 18 may include any appropriate materials. For example, the coating 18 may include, but is not limited to, biologically active materials such as growth hormones, genetic materials, antibodies, cellular matrices, or other appropriate bioactive materials. In addition, the coating 18 may include bioactive materials that may be released and absorbed into an anatomy after implantation.

In addition, the coating 18 may include biocompatible materials that may be selected for reasons other than providing a bioactive material to the anatomy. For example, various hydrogels, collagens, bio-absorbing polymers, and microcellular foams may be provided in the coating 18. It will be understood that any or all appropriate bioactive materials may be provided in the coating 18. Therefore, the coating 18 may include one or a plurality of portions to provide a selected bioactivity, bioaction, or other appropriate portion. For example, the coating 18 may include an antibiotic to assist in an anatomy's reception of the beads 12. One example would include, but is not limited to, antibodies to endothelial cells. In addition, the coating 18 may include a hydrogel which may swell or expand in volume in an anatomical location. As discussed herein, the hydrogel in the coating 18 may expand to increase the volume of the beads 12 for a selected purpose.

The beads, such as the first bead 12a and the second bead 12b, may be interconnected with a selected portion. For example, the beads 12 may be provided on a strand 20. The strand 20 may substantially pass through the beads 12 such that the beads, such as the first bead 12a and the second bead 12b, are operable to move relative one another.

The strand 20 of the beads 12 may be formed of other appropriate materials, in particular the strand 20 that interconnects the plurality of beads may be formed of any appropriate material. Various materials include therapeutic agents, copper or copper alloys or a variety of any other therapeutically active metals, alloys, or components. In addition, various fibers may be used to form the strand 20 such as a polyester (Dacron™), polyglycolic acid, polylactic acid, fluoropolymers, nylon, polyaramids (e.g., Kevlar®) or silk chosen for any selected purpose such as thrombogenicity.

In addition, the strand may be formed of a cable or braided material, such as representing a micro cable wherein each of the fibers of the strand may be formed of one or more of the selected materials, such as those discussed above, or any other appropriate materials. In addition, one or more of the strands may be shorter than the others such that they are intermittently terminated to extend beyond the diameter of the strand 20. This may increase the therapeutic aspect of the strand 20 in addition to the beads 12 of the occluding device 10.

The strand 20 may be coated, in addition to the beads 12 being coated with the coating 18, with a selected material or be impregnated with a selected material. Various materials may include therapeutic materials that include, but are not limited to, one or more of a human growth hormone, other genetic materials, antigens, hydrogels, collagen, bioabsorbable polymers, including, but not limited to, lactic acid, glycolic acids, caprolactam or microcellular foam. It will also be understood that the strand 20, either alone or in combination with the beads 12, may be used to transmit a therapeutic element, such as energy, therefore the strand 20 may also include an energy conductor such as metal or a fiber optic material. Moreover, it will be understood that the strand 20 may be formed in any appropriate manner such as forming a bundle or constrained cable. It will be understood, as discussed herein, that the strand 20 may also be formed of strand segments. It will be understood that any appropriate strand segment may include a selected configuration while other strand segments include other selected configurations. Therefore, it will be understood that the strand of the occluding device, according to various embodiments, may be formed for various reasons such as thererapeutic elements, therapeutic transmission, strength, or flexibility.

Alternatively, the beads 12 may be fixed to the strand 20 such that the beads are substantially immobile relative to an adjacent bead yet the strand 20 is substantially flexible to allow for movement of the strand 20 relative to a selected portion of the anatomy. Furthermore, the strand 20 may be a substantially independent plurality of segments that are interconnected between adjacent beads, such as the first bead 12a and the second bead 12b to allow for a substantial plurality of beads to be formed next to one another yet to allow for a substantial flexibility of the complete occluding device 10 to allow for ease of implantation and positioning. Nevertheless, the beads 12 are generally provided at a selected distance B between adjacent beads. The distance B may be generally about equal to the diameter A of the beads 12. Therefore, the distance B may be about 0.01 inches to about 0.02 inches (about 0.25 mm to about 0.51 mm). Although the distance B may be any appropriate distance, such as a distance less than or more than the diameter A of the beads 12. Nevertheless, as mentioned above, the beads may be allowed to move relative to one another such that the distance B is not substantially constant. Nevertheless, the distance B may be provided as a maximum distance that the beads 12 are able to move adjacent to one another.

With reference to FIG. 1B, an occluding device 10b is illustrated. The occluding device 10b may be similar to the occluding device 10 except that it further includes or is formed with a coil 21 that surrounds at least a portion of the beads 12. Thus the beads 12 need not be positioned alone, but may be positioned with the coil 21. Both the coil 21 and the beads 12 may include a selected material, such as a hydrogel.

Therefore, the occluding device 10, 10b, according to various embodiments, may provide a plurality of beads 12 that are operable to be provided on the strand 20. The strand 20 may be a substantially continuous or discontinuous strand to provide the occluding device 10 to be easily implanted into a selected portion of the anatomy. With reference to FIG. 2, the occluding device 10, 10b may include a plurality of the beads 12 (and/or the coil 21) to substantially form the occluding device 10 at a selected length. Nevertheless, due to the positioning or formation of the strand 20, the occluding device 10 may be substantially flexible such that a plurality of shapes or configurations, such as two dimensional or three dimensional configurations, may be produced with the occluding device 10.

The occluding device 10, or according to any appropriate embodiment, may be substantially flexible such that it may be provided in substantially any shape, orientation, volume filling geometry or the like. In addition the coil 21 may also be flexible to allow for forming the occluding device 10b in any appropriate configuration. For example, the beads 12 are positioned on the strand 20 such that the occluding device 10 may be moved to any appropriate geometry or volume with substantial efficiency. Therefore, the occluding device 10 may be used to fill substantially any volume, such as a vascular region, aneurysm, or the like, with efficiency and relative quickness.

The occluding device 10 may include any selected length depending upon the number of beads 12, the length of the strand 20 or any other appropriate consideration. Nevertheless, the plurality of the beads 12 provides for the occluding device to include any appropriate length. In addition, the distance B between respective beads 12, may allow for a severing of the occluding device 10 and a selected interbead area to allow for the substantially interoperative or customization of a length of the occluding device 10.

With reference to FIG. 3, according to various embodiments, an occluding device 40 may be provided that includes a bead 42 or a plurality of beads 42. The beads 42 generally include an exterior surface 44 and an interior surface 46 that substantially defines an internal void or opening 48. The beads 42, therefore, include a wall or hollow member that substantially defines the internal void 48. The strand 20 is provided as a plurality of segments 52 that substantially terminate within the void 48.

Each of the strand segments 52 may terminate in an obstructing portion 54 that is formed within the void 48, but not able to pass through an opening or strand passage 56 defined by the bead 42. Because the strand passage 56 is generally larger than a diameter or dimension C of the strand segment 52, the strand segment 52 is operable to move relative to each of the beads 42 generally in a direction of arrow D. Therefore, the bead 42 may move in the direction of arrow D or the strand segment 52 is operable to move in the direction of arrow D. Nevertheless, the strand segment 52 is not able to generally move more than the diameter of the void 48 before the obstructing portion 54 reaches the interior surface 46. This allows each of the beads 42 to move relative to an adjacent bead or even other beads on the occluding device 40, therefore allowing for a substantial flexibility, yet the beads 42 are still moveable or flexible relative to selected adjacent beads.

It will be understood that the beads 42 may also include a coating such as the coating 18 on the beads 12. In addition, the occluding device 40 may be similar to the occluding device 10 in that a plurality of the beads 42 may be provided in any appropriate length. In addition, the strand segment 52 may be cut or severed at a selected area between two beads to allow for a customization of a length of the occluding device 40. In addition, each of the portions including the beads 42 and the strand segments 52 may be formed of any appropriate material. Nevertheless, the materials may generally be selected to be substantially radio-opaque for easy viewing using a selected imaging device. It will be understood that providing the beads 42 in a radio-opaque manner may allow for easy viewing after implantation of the occluding device 40 into a selected application.

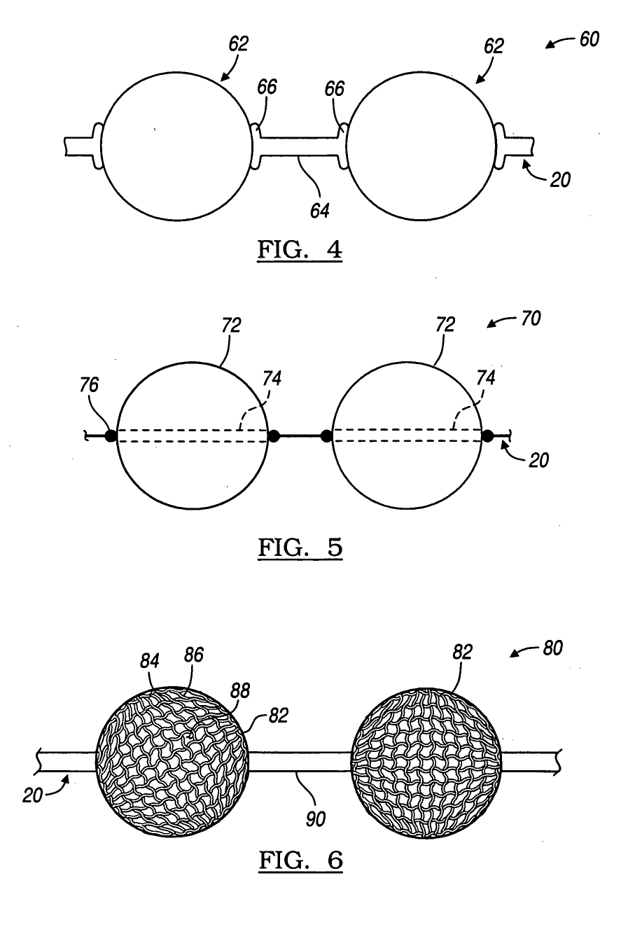

With reference to FIG. 4, according to various embodiments, an occluding device 60 is illustrated. The occluding device 60 may include one or a plurality of beads 62. The beads 62 may be substantially solid such as the beads 12 or may be substantially hollow such as the beads 42 or “caged” such as the beads 82, described below. Nevertheless, the strand 20 may include a plurality of strand segments 64 that are provided between the plurality of beads 62. The strand segment 64 may be adhered or fixed to an exterior surface of the beads 62. An affixation or weld portion 66 may substantially permanently hold the strand segment 64 relative to a selected one or two of the beads 62. Because of the size of the beads 62 and the strand segment 64, the occluding device 60 may be provided in a selected length, such as the occluding device 10 and still be substantially flexible. In addition, it will be understood that the beads 62 may include any appropriate coating, such as the coating 18 and may be formed of any appropriate material. Therefore, the occluding device 60 may also be substantially radio-opaque and provided through a selected cannula for implantation, as discussed herein.

It will be understood that substantially solid exterior surface beads 12, 42, and 62 may be provided in any appropriate manner on a strand 20. Therefore, according to various embodiments, including those exemplary embodiments discussed above, the beads may be provided on the occluding devices 10, 40, and 60 for implantation into a selected area or region. In addition, the beads may be formed in any appropriate manner for various reasons or selection by a user. Moreover, various embodiments may be provided for substantial ease of implantation of the occluding devices 10, 40, and 60 or may be provided for a substantial rigidity, strength, or the like. Nevertheless, the embodiments of the occluding devices 10, 40, and 60 are merely exemplary and not intended to be limiting. In addition, each of the occluding devices 10, 40, and 60 may be provided for various reasons, such as the exemplary embodiments discussed herein.

With reference to FIG. 5, an occluding device 70, according to various embodiments, is illustrated. The occluding device 70 may include a plurality of beads 72 of any appropriate configuration, including those discussed above and herein. The beads 72 are substantially interconnected with the strand 20. The strand 20 may be any appropriate material, such as those discussed above and for example including a suture strand that may be formed of any appropriate material. For example, the strand 20 may be formed of a polymer such as aeromoline or a metal, either of which may be radio-opaque for viewing using an imaging device. Regardless, the strand 20 substantially interconnects a plurality of the beads 72 by passing through each of the beads 72. Therefore, each of the beads 72 may define a strand passage 74 to allow the strand 20 to pass through the respective beads 72.

The strand 20 may be knotted or otherwise fixed to include a stop member 76 positioned near the bead 72 at the end of the strand passage 74. Therefore, the knot or other stop member 76 may stop movement of the bead 72 relative to the other of the beads 72 or the strand 20. The knot or stop member 76 may be any appropriate portion. A knot may be used if the strand 20 is substantially flexible or a weld or solder portion may be used if the strand 20 is formed of a selected metallic material. Regardless, it will be understood that the knot or stop member 76 may be provided to hold the beads 72 relative to the strand 20.

Holding the beads 72 relative to the strand 20 may allow for an easy implantation of the occluding device 70. That is the occluding device 70 may be passed through a selected instrument, such as cannula, without substantially knotting or bending the occluding device 70. Because the beads 72 are held relative to the strand 20, the beads 72 are resistant to bunching relative to one another during movement of the occluding device 70. Therefore, the occluding device 70 may be easily moved into a selected portion, such as a vascular portion, without bunching the beads 72 together so that they may be easily spaced and positioned for a selected purpose.

It will be understood that the beads 72 may be formed in any appropriate manner. The beads 72 may be substantially solid, may be substantially hollow as discussed above or caged as discussed below. In addition, the beads 72 may be formed of any appropriate material, such as a polymer or metal or metal alloy. Therefore, the beads 72 may be substantially radio-opaque if selected, for viewing with an imaging device after implantation of the occluding device 70 into a selected anatomical portion.

With reference to FIG. 6, an occluding device 80 is illustrated. The occluding device 80 may include a bead or plurality of beads 82. The beads 82 may be formed as a substantially hollow or open cage. The beads 82 may be understood to be generally porous or formed of a porous material. Thus material positioned in the beads 82 may pass to the exterior or an external environment may affect the material positioned in the beads 82.

The beads 82 may be made porous in any appropriate manner. For example, a first set of strands 84 may be woven about a plurality of second strands 86 that are formed at an angle relative to the first set of strands 84. Therefore, the beads 82 may be formed in any appropriate structure, such as a substantially three dimensional sphere structure including a plurality of the intersecting or woven strands 84, 86. The strands may form a selected structure that defines a hollow interior 88. The hollow interior 88 may be accessed through pores defined by the woven strands 84, 86 such that a material positioned within the hollow interior 88 may exit the beads 82 or be affected by an external environment. Alternatively, the bead 82 need not be woven and simply may include punched or formed bores in a surface.

Whatever configuration is utilized, having a smooth exterior surface such as but not limited to metal, reduces the frictional force generated during deployment of the device and reduces the risk of thromboembolic complications compared to devices that have embolic material coated on their exterior surface. In other words, during deployment, the smooth exterior surface of each of the functional units described herein will rub against each other rather than contacting embolic material as occurs with current embolic devices that are “coated” with embolic material such as hydrogel. In this manner, there will be a reduction in the frictional force generated while the device described herein interacts with its functional units during deployment as well as a lower incidence of particulate debris resulting from embolic material rubbing against itself which can result in thromboembolic phenomenon or stroke (when used intracranially). Again, this is because in the present invention, the embolic material is contained within the functional units and not on the exterior surface of the functional units.

The beads 82 may be formed of any appropriate material that allows it to obtain a selected structure. For example, the strands 84, 86 may be formed of a substantially rigid metal or polymer material, but not limited thereto. The strands 84, 86 may also be formed of a substantially radio-opaque material such that they may be viewed with an imaging device after implantation into an anatomy. Regardless, the strands 84, 86 are operable to define the hollow interior 88 in which a selected material may be positioned for a bioactivity or biopurpose.

The substantially hollow or open cage beads 82 may be formed in any appropriate manner, in addition to those discussed above and according to various embodiments. For example a bead may be formed by, but not limited to, including an internal structure over which a netting or meshed material is placed. For example, a hollow column material may be formed to include a plurality of slits such that a column force may expand the size of the slitted column member to expand in a substantially ellipsoidal or spherical manner. The expanded column member may then have a mesh placed around the expanded portion. The expandable portion may be formed of a shaped memory material such that the substantially spherical shape may be formed as the memory shape so that it may be deformed and reobtain the selected or expanded shape. Various appropriate shape memory materials may be used such as, but not limited to, a nickel titanium alloy such as Nitanol™ or shape memory polymers. Various formations of a mesh cover or porous member may include those described in U.S. Pat. No. 6,428,558, incorporated herein by reference.

According to various embodiments, the hollow interior 88 may substantially include a selected material, such as those discussed above for the coating 18 or any other appropriate material. For example, the material positioned in the hollow interior 88 may be a hydrogel, but not limited thereto, that is allowed to substantially expand after implantation into an anatomical structure. Regardless, the material positioned in the hollow interior 88 may be provided for any appropriate purpose.

If the material positioned in the hollow interior 88 is provided as a hydrogel, the material inside of the hollow interior 88 may expand many times after implantation of the occluding device 80. As discussed herein, hydrogel or a bioactive polymer may increase its volume in any appropriate dimension and amount depending upon the selected material. Therefore, because a substantial entirety of the volume of the beads 82 may be filled with the selected material, due to the fact that the strands 84, 86 substantially define only an exterior surface of the beads 82, the material positioned in the hollow interior 88 may be allowed and supplied in a selected volume to expand many times greater than the size and volume of the beads 82. Therefore, even if the beads 82 include a diameter, such as that discussed above, providing hydrogel in the hollow interior 88 may substantially increase the occluding power of the beads 82.

The beads 82 may be provided on the strand 20. As discussed above, the strand 20 may be any appropriate strand or formed of any appropriate material. For example, the strand 20 may include a plurality of strand segments 90 that substantially interconnect a plurality of the beads 82. The strand segment 90 may be fixed to an exterior of the beads 82 such as to a portion of the strands 84, 86. Alternatively, as discussed above, the strand segment 90 may be provided to be substantially mobile relative to the beads 82 or be substantially immobile relative to the beads 82. Regardless, the beads 82 may be implanted in an appropriate manner such that the beads 82 are provided relative to a selected portion of the anatomy to allow for a material that may be positioned in the hollow interior 88 to provide a selected bioactivity.

With reference to FIG. 6, the occluding device 80 that includes the beads 82, as discussed above, substantially defines a hollow cage bead. It will be understood that the cage that the beads 82 define substantially include a plurality of pores that allows the hollow interior 88 to be filled with a material that is able to pass through the pores defined by the strands 84, 86 of the beads 82.

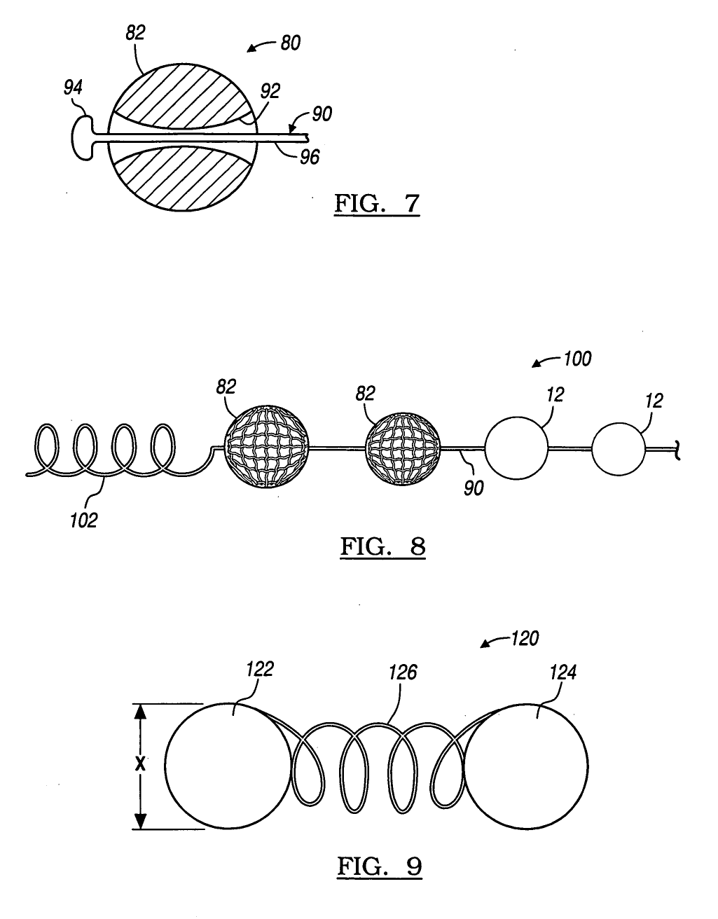

In addition, with reference to FIG. 7, the beads 82 may define a strand passage 92 that allows the strand segment 90 to be positioned therein. The strand passage 92 may be defined on the interior of the beads 82 such that the strand segment 90 may substantially interconnect the beads 82 with one or more of a plurality of the beads 82. The strand segment 90 may include a bead engaging portion 94 and a body or length portion 96. Therefore, the strand segment 90 including the bead engaging portion 94 may engage a portion of the passage 92 substantially defined by the bead 82.

The passage 92 may form a narrow or engaging region that allows for the engaging portion 94 of the strand segment 90 to engage a portion of the beads 82 such that the strand segment 90 only moves a selected distance relative to the beads 82. It will be understood that this mechanism is merely exemplary, and a plurality of mechanisms may be provided to hold the beads 82 relative to a strand to form the occluding device 80. Regardless, to provide a selected amount of flexibility, it may be desirable to provide the strand as a plurality of the strand segments 90. Therefore, the beads 82 are operable to move relative to the strand segment 90 to provide a flexibility of the occluding device 80 to allow for manipulation of the occluding device 80 during implantation thereof.

Therefore, the occluding device 80 may be provided with portions that allow for an efficient implantation of the occluding device 80 and placement of the occluding device 80. The flexibility of the occluding device 80 may allow for positioning the occluding device 80 without substantially binding the occluding device and inhibiting positioning thereof. This may increase the efficiency of the implantation into the aneurysm to occlude or obstruct the aneurysm or the parent artery and obstruct the aneurysm lumen.

The occluding device according to various embodiments, such as those illustrated above, may also include other portions in addition to one or a plurality of beads. For example, with reference to FIG. 8, an occluding device 100 may include one or a plurality of the beads 82. It will be understood that the beads 82 may be any appropriate bead according to various embodiments. Regardless, the beads 82 may be interconnected with the strand segment 90 to allow for a substantially flexibility of the occluding device 100 during an implantation thereof. Nevertheless, the occluding device 100 may include a coil or other geometrical configuration portion 102 to replace one or more of the beads 82 and be substantially affixed or attached to one of the strand segments 90. In addition, the coil 102 may be connected directly to the bead 82. Therefore, the coil 102 may be a portion of the strand or viewed as a portion of the bead 82. Moreover, if the coil 102 is attached to the bead 82, it may be attached in any orientation commonly known in the art. The coil may be attached in such a manner that it represents the proximal portion of the embolic device, or the distal or trailing portion. The coil may represent a “helical” two-dimensional or a three-dimensional coil configuration. Also, the coil 102 may be attached anywhere on the occluding device 100, or any of the various embodiments.

The coil 102 may be any appropriate coil, such as those known in the art, and disclosed in U.S. Pat. No. 6,238,403, incorporated herein by reference. Regardless, the coil 102 may provide a mechanism to terminate the occluding device 100 to allow for a substantial obstructing of an aneurysm or occluding of a vascular system. Therefore, the occluding device 100 may be implanted to a selected position or purpose, such as to obstruct an aneurysm and the coil 102 may provide a generally known application for occluding a vascular region or obstructing an aneurysm. Thus, the occluding device 100 may incorporate both the beads 82, or any other appropriate bead according to various embodiments, and the coil 102.

In addition, the occluding device 100 may also include beads that are not exactly similar to one another. The occluding device, according to various embodiments, may include a plurality of types of beads. As discussed above, the beads may be provided in a plurality of configurations, such as substantially solid beads or beads that are substantially porous such that a material may be positioned within the bead and allowed to exit from the bead at a selected time. Therefore, the occluding device may include a plurality of beads depending upon a selected operation or use. For example, a selected amount of hydrogel may be provided that may be finely tuned by selecting beads of various configurations to form the occluding device. Therefore, the occluding device 100 may include the beads 82, that are substantially porous, and the beads 12 that are substantially solid and include a substantially continuous surface. This allows the occluding device 100, according to various embodiments, to be formed according to any selected specifications for forming the occluding or obstructing device.

An occluding device 120 may include a first bead 122 and a second bead 124 substantially interconnected with a coil 126. The occluding device 120 may be substantially provided as a single unit that is not interconnected with other units. Alternatively, the occluding device 120 may define a single unit or a plurality of units that are interconnected. The occluding device 120 includes the beads 122 and 124 according to various embodiments. As discussed above, the beads 122 and 124 may be substantially solid or define a continuous surface or be substantially porous. In a porous bead, as discussed above, the hollow interior of the bead may be filled with a selected material to interact with the anatomy. Therefore, the single unit, or occluding device 120 may include a selected amount of a material, such as hydrogel to expand to a selected volume.

The occluding device 120 includes the coil 126 to provide a single unit occluding device which may be positioned to occlude a vascular region or to obstruct an aneurysm. Therefore, the occluding device 120 may be provided for selected sizes of aneurysms that may be substantially obstructed with a single occluding unit. Alternatively, the occluding device 120 may be provided to obstruct or occlude a selected portion of the aneurysm. It will be understood that the occluding device 120 may be used in conjunction with an occluding strand, such as the occluding device 10 or 100, or any appropriate occluding strand according to various embodiments, to occlude a vascular region or obstruct an aneurysm.

The occluding device 120 may substantially define a selected dimension X, such as a diameter, width, height or any other appropriate dimension. Regardless, the dimension X may allow that the occluding device 120 may be positioned and operated as a substantially single unit for positioning relative to a vascular region or an aneurysm. The dimension X may be any appropriate size, such as those discussed above. Therefore, the dimension X may be about 0.05 inches to about 0.02 inches (about 0.25 mm to about 0.51 mm). The dimension X may allow for the occluding device 120 to be positioned with a selected instrument, such as a microcatheter for any appropriate purpose.

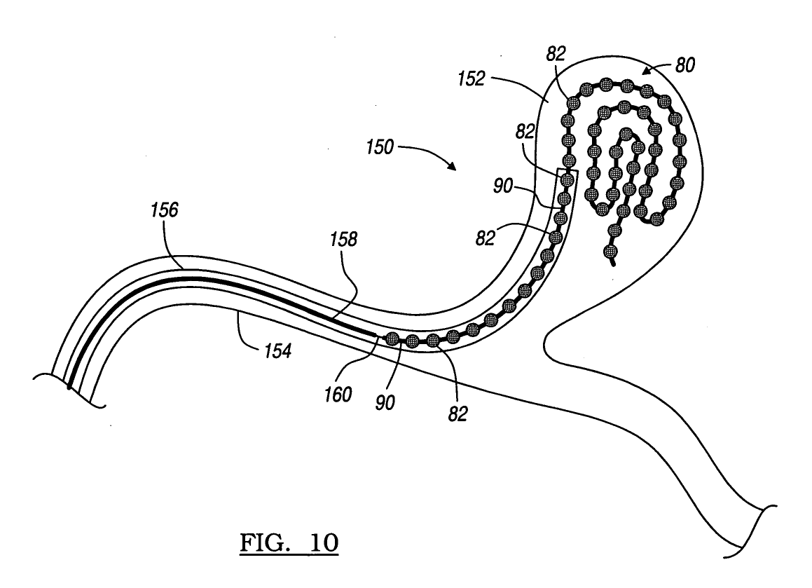

The occluding strands or devices, according to various embodiments, such as those exemplary illustrated above, may be implanted according to any appropriate mechanism. It will be understood that the various occluding devices may be implanted using a catheter to allow for passing the occluding device or portion relative to a selected portion of the anatomy. With reference to FIG. 10, an anatomical portion 150 may include an aneurysm or aneurysm lumen 152. The aneurysm 152 may extend from a vascular region or vessel 154.

The aneurysm 152 may be positioned or occur in any portion of the anatomy. For example, the aneurysm 152 may be substantially intracranial such that it occurs within a portion of the brain or intracranially. As is generally known in the art, an intracranial aneurysm may inflict upon a patient an undesirable condition especially if the aneurysm 152 continues to fill with flow from the vascular region 154. The aneurysm 152 may burst and substantially injure the patient. Therefore, it may be desirable, as discussed above, to occlude the flow to or obstruct the aneurysm 152.

The obstructing device or occluding device 80, or the obstructing device according to any appropriate embodiment, may be provided to the aneurysm 152 through a selected catheter or microcatheter 156. The catheter 156 may be passed through the vascular region 154 as is generally known in the art. The catheter 156 may be substantially guided or navigated to a selected position, such as within the aneurysm 152. The catheter 156 may allow for positioning the occluding device 80 relative to a selected portion of the aneurysm 152 to allow for substantially obstructing the aneurysm 152 to allow for stopping a flow of the blood or material to the aneurysm 152 to possibly eliminate the aneurysm 152 from possibly continuing to grow, perforate or rupture.

The catheter 156 may be formed of a selected diameter such as one that substantially allows for passing of the occluding device 80 therethrough. As discussed above, the occluding device 80 may include the beads 82 of any selected diameter. Therefore, the catheter 156 may be dimensioned to allow for passing the occluding device 80 through the catheter for substantially positioning the occluding device 80. It will be understood that the catheter 156 may include an internal diameter substantially complimentary to an external diameter of the beads 82. Furthermore, the beads of the occluding device according to various embodiments, may be coated on an exterior surface with a selected material. Therefore, the catheter 156 may be dimensioned to allow for passing the occluding device, with beads that include a selected exterior coating, without substantially disturbing the coating on the beads.

The occluding device 80 may be any appropriate occluding device, such as those discussed above. It will be understood that the occluding device 80, including the spheres or beads 82, that include the hollow interior 88, are merely exemplary. Regardless, the beads 82 that are interconnected with the strand segments 90 may be positioned substantially flexibly within the aneurysm 152. Because the occluding device 80 includes a portion or plurality of portions that are interconnected, the occluding device 80 may be substantially flexible to allow for an efficient positioning and easy manipulation of the occluding device 80 within the aneurysm 152.

The catheter 156 may be used to position and move the occluding device 80 during the implantation to allow for a selected positioning of the occluding device 80 in a substantially complete obstruction of the aneurysm. Moreover, the occluding device 80 may be provided in a manner that allows for forming a selected geometry within the aneurysm 152 to provide for a substantially complete and efficient obstruction of the aneurysm 152.

In addition, as discussed above, the beads 82 may be filled or include a selected material, such as hydrogel, that may swell or expand upon introduction into the biological atmosphere. The hydrogel, or any appropriate material, may be selected to swell at a rate that allows for positioning of the occluding device 80 in a substantially efficient manner and does not swell until the occluding device 80 is positioned. Nevertheless, the amount and consistency of material positioned within the beads 82 may allow for swelling that substantially increases the volume of the occluding device 80 over the volume defined by the plurality of the beads 82. Therefore, the substantial flexibility of the occluding device 80 may allow for positioning the occluding device 80 using the known expansion of the hydrogel, or any appropriate material, to select or create an obstruction within the aneurysm 152 according to a selected operation to substantially ensure an efficient and complete obstruction.

The occluding device 80 may be passed through the catheter 156 with a pusher or moving wire 158. The pusher 158 may be formed of any appropriate material, such as stainless steel, titanium, a polymer or any appropriate material. Regardless, the pusher wire or strand 158 allows for pushing the occluding device 80 from the catheter 156 into the aneurysm 152. The pushing wire 158 may be substantially manually or automatically operated to push the occluding device 80 from the catheter 156 into the aneurysm 152. Also, as discussed above a detachment point 160 may be provided to remove the occluding device 80 from the pusher wire 158. The detachment point 160 may be an area where an electrical charge may separate the wire or any other appropriate mechanism may be used. Regardless, the pusher wire 158 may be separated from the occluding device 80. It will also be understood that the detachment point may be positioned at any appropriate location or there may be more than one of the detachment points 160.

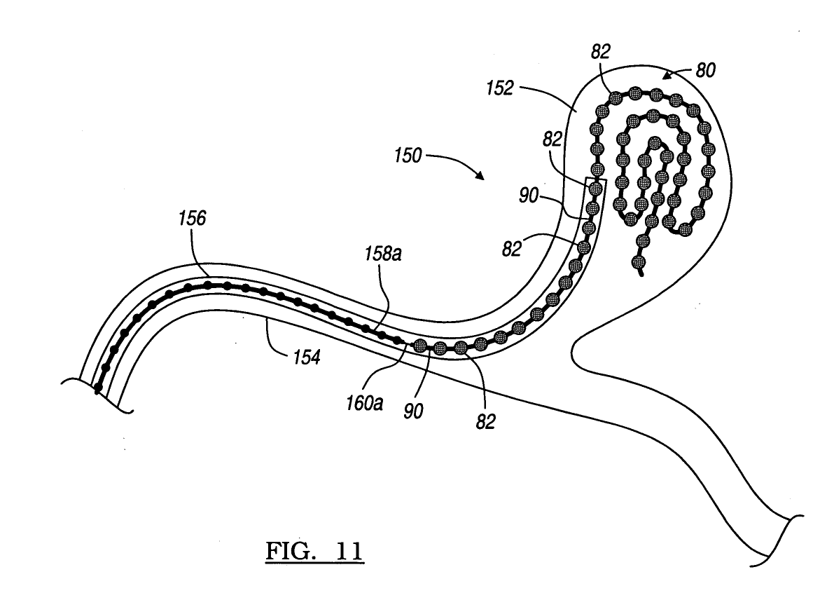

As illustrated in FIG. 11, a pusher wire or member 158a can be made in the same or similar “beaded configuration” as the embolic occlusion devices discussed herein according to various embodiments. This allows for the embolic device 80 to be advanced in the catheter 156 with decreased rigidity. Often, the catheter 156 makes several twists and turns prior to gaining access to the aneurysm. A “stiff” pusher wire attached to the endovascular coil or embolic device 80 may make it more difficult to advance such a device into an aneurysm. Having multiple break points with the beaded configuration pusher wire 158a, allows the pusher wire 158a to have decreased rigidity and greater potential for advancing coils or embolic devices into an aneurysm surrounded by tortuous vasculature. A detachment point 160a, similar to that of detachment point 160 may also be provided.

The occluding device 80 may be disconnected from the pusher wire 158 in any appropriate manner at the detachment point 160. For example, the pusher wire 158 may be substantially electromechanically disconnected from the occluding device 80 by passing a selected current through the pusher wire 158 that substantially disconnects the pusher wire from the occluding device 80. Therefore, once a selected amount of the occluding device 80 is positioned within the aneurysm 152, the pusher wire 158 may be disconnected from the occluding device 80.

Any other appropriate disconnecting mechanism may be provided to disconnect the occluding device 80 from the pusher wire 158. Moreover, it will be understood that the occluding device 80 may be disconnected at any appropriate portion when a selected amount of the occluding device 80 is positioned within the aneurysm 152. A selected length or volume of the occluding device 80 may be substantially preselected using a known size of the aneurysm 152 that may be determined using various imaging techniques.

Alternatively, the volume of the occluding device 80 may be selected substantially intra-operatively. A selected number of the beads 82 may be positioned in the aneurysm 152 by severing one of the plurality of the strand segments 90 between adjacent beads 82 to provide only a selected number of the beads 82 into the aneurysm 152. Therefore, a plurality of the beads 82 may be provided in a selected occluding device 80 and only a selected number or volume of the beads 82 be passed into the aneurysm 152 depending upon a selection by a user, such as a surgeon.

Therefore, it will be understood that occluding devices according to various embodiments may include a plurality of beads or occluding portions that may be interconnected on a strand or with a plurality of strand segments. The occluding device 80 may be substantially flexible to allow for positioning the occluding device in a selected manner in the aneurysm 152. This may allow for positioning the occluding device 80 in a manner that allows for occluding or obstructing the aneurysm 152 in a selected manner. In addition, due to the flexibility of the occluding device 80, the occluding device 80 may be positioned in a substantially selective manner rather than being limited by the rigidity or inflexibility of the occluding device. Moreover, the selectability of the volume to be positioned may allow for easy positioning of the occluding device within the aneurysm 152 to achieve a selected result, such as decreasing or limiting flow to the aneurysm 152 to allow for substantial healing or elimination of an expansion of the aneurysm 152.

Occluding devices, according to various embodiments, may be formed in any appropriate manner, such as, but not limited to, the embodiments discussed above, or any appropriate variations. The occluding devices according to various embodiments may be formed in a substantially flexible manner. As discussed above, the beads according to various embodiments may be formed of a selected diameter or size and interconnected with a strand or strand segment that allows the entire occluding structure or device to be substantially flexible overall. As discussed above, an occluding device that includes a selected length and a substantially small diameter may provide a substantially flexible device so that it may be easily used to selectively or randomly fill a selected volume with the device. For example, including a substantially small diameter may provide the occluding device as a device that is substantially similar to a rope or thread of a substantially small diameter that is easily flexible and efficiently positioned in a selected volume.

In addition, the selected size of the occluding device may allow it to be implanted without a substantially rigid or integrated pushing or implanting portion. For example, the occluding device may be of a size that allows for a substantially fluid implantation. That being, the occluding device is carried in a fluid carrier for implantation into a selected position. The fluid carrier may be any appropriate material such as a pharmeutical or a substantially sterile material. Providing such an implantation may allow for efficient positioning of the occluding device in a position.

Regardless, the structure or the size of the occluding device may allow for a substantially flexible occluding device that may be efficiently and with little force provided in, but not limited to, any appropriate shape, geometry, volume and the like. Moreover, the substantial flexibility of the occluding device may substantially eliminate kinking or undesirable shape or geometry formations of the occluding device during implantation.

In addition, the beads may be formed according to a plurality of specifications that allow for positioning a selected amount of the material, such as a bioactive material, relative to the aneurysm 152. Therefore, the beads may include a hydrogel or swelling material that may be included to a selected volume with the bead, such as coated on an exterior thereof or substantially filling the bead. Therefore, the bead may swell, because of the hydrogel, according to any selected manner depending upon a volume of the hydrogel provided with the bead. In addition, any other appropriate bioactive material may be provided with the bead or occluding portion. The hydrogel, various other bioactive ingredients, including but not limited to, genetic material or solutions, medicinal products, cellular matrix materials, and the like may be provided with the occluding portion, such as the bead, in the occluding device. Therefore, the occluding device may be provided to position a selected portion of a bioactive material in the aneurysm 152 in addition to providing the occluding mechanism.

Also, the occluding mechanism allows for a substantially intra-operative selection of a volume of occlusion or obstruction. Therefore, rather than providing a plurality of individual members that may be used to fill or selectively obstruct a portion of the anatomy, such as the aneurysm 152, the positioning of a volume of the occluding device may be performed substantially intra-operatively. This may allow for a selection of occlusion or obstruction of the aneurysm 152 during an operative procedure. Thus, the occluding device allows for an intra-operative selection without requiring the positioning of a plurality of individual portions through a selected mechanism, such as the catheter 156 into the aneurysm. As discussed above, the mechanism for selecting the size of the occluding device may be performed in any appropriate manner, such as electromechanical, mechanical or the like. Thus, it will be understood that the occluding device may be formed to the selected portion of the anatomy to provide for occluding or obstructing the aneurysm 152 to restrict a flow through the vascular region 154.

The description of the invention is merely exemplary in nature and, thus, variations that do not depart from the gist of the invention are intended to be within the scope of the invention. Such variations are not to be regarded as a departure from the spirit and scope of the invention. Moreover, although several exemplary embodiments are illustrated according to various embodiments it will be understood that various combinations, not specifically illustrated or discussed may be provided according to various embodiments.

1. An embolization system comprising:
a flexible conduit;
a plurality of spherical functional units disposed at spaced intervals along said flexible conduit; and
a hydrogel solution carried by each of said plurality of spherical functional units.

2. The embolization system of claim 1 wherein each of said spherical functional units comprises a wire-frame cage structure, said wire-frame cage structure defining an internal cavity.

3. The embolization system of claim 2 wherein said internal cavity is adapted to receive said hydrogel solution.

4. The embolization system of claim 1 wherein said spherical functional units comprise a solid metallic ball.

5. The embolization system of claim 4 wherein said solid metallic ball is coated with said hydrogel solution.

6. The embolization system of claim 1 wherein said spherical functional units comprise a hydrogel foam structure.

7. The embolization system of claim 1 wherein said conduit includes a length between about 0.39 inches and about 19.69 inches (10 mm and 500 mm).

8. The embolization system of claim 1 wherein said spherical functional units include an outside diameter substantially equal to about 0.018 inches (0.46 mm).

9. The embolization system of claim 1 wherein said spherical functional units include an outside diameter substantially equal to about 0.014 inches (0.36 mm).

10. The embolization system of claim 1 wherein said flexible conduit includes at least one detachment zone, said at least one detachment zone operable to allow said flexible conduit to separate at said at least one detachment zone to achieve a desired length of said flexible conduit.

11. An embolization system comprising:
a plurality of spherical functional units, said plurality of spherical functional units each defining an internal cavity;
a flexible conduit fixedly supporting said plurality of spherical functional units, said flexible conduit extending through said internal cavity of each of said spherical units; and
a hydrogel solution disposed within said internal cavity of each of said plurality of spherical functional units.

12. The embolization system of claim 11 wherein said spherical functional units include a surface defining each of said spherical functional units.

13. The embolization system of claim 12 wherein said surface defines a pore;
wherein said hydrogel solution is operable to exit said internal cavity.

14. The embolization system of claim 11 wherein said hydrogel solution is substantially hydrophilic and operable to expand when exposed to an environment.

15. The embolization system of claim 11 wherein said spherical functional units include a diameter of about 0.01 inches (0.25 mm) to about 0.02 inches (0.51 mm).

16. The embolization system of claim 11, further comprising:
a coil unit.

17. The embolization system of claim 16 wherein said coil is coated with said hydrogel solution.

18. The embolization system of claim 11 wherein said plurality of spherical functional units are substantially positionable relative to each of the other of said spherical functional units.

19. An embolization system for substantially obstructing an aneurysm, comprising:
a first member disposable relative to the aneurysm;
a second member disposable relative to the aneurysm; and
a connection member interconnecting said first member and said second member;
wherein said first member and said second member are moveable relative to one another via said connection member for implantation of said first member and said second member.

20. The embolization system of claim 19 wherein said first member and said second member are substantially similar and selected from a group comprising a hollow member, a solid member, a porous member, and combinations thereof.

21. The embolization system of claim 19 wherein said first member and said second member include a plurality of said first and said second members interconnected to substantially form a flexible system to substantially fill a volume.

22. The embolization system of claim 19 wherein at least one of said first member and said second member include a substantially porous surface, wherein a material positioned in said first member or said second member is exposed to an environment in which said first member or said second member are placed.

23. The embolization system of claim 22 wherein said porous surface is substantially defined by a woven strand forming a woven configuration such that a surface of said first member or said second member includes a pore defined by said woven strand; wherein said pore is operable to expose an interior portion of said first member or said second member to an environment.

24. The embolization system of claim 22 wherein a hydrogel is included in said first member or said second member; wherein said hydrogel expands at a selected rate upon introduction into an anatomical position.

25. The embolization system of claim 19 wherein said connection member interconnects a plurality of said first members and said second members, and said connection member substantially passes through each of said plurality of said first members and said second members.

26. The embolization system of claim 19 wherein said connection member interconnects said first member and said second member by passing through said first member and said second member, such that said first member and said second member are substantially moveable relative to said connection member and each other.

27. The embolization system of claim 19 wherein said first member and said second member includes an external geometry selected from a group comprising a sphere, a rectangle, an octagon, an oval, or combinations thereof.

28. The embolization system of claim 19, further comprising a bioactive material.

29. The embolization system of claim 19 wherein said first member or said second member include an external dimension of about 0.01 inches (0.25 mm) to about 0.02 inches (0.51 mm).

30. A method of providing an embolization system to a vascular region including an expanding element, comprising:
providing a package including a volume;
placing an expanding element in said volume;
providing a bore in said package such that said expanding element is operable to interact with an environment surrounding said package; and
guiding said package to the vascular region.

31. The method of claim 30 wherein providing said package includes:
forming a substantial sphere from a member that is woven to form said bore between portions of said member;
wherein said woven sphere defines said volume.

32. The method of claim 30 wherein placing an expanding element includes providing a hydrogel operable to expand at least six times in volume in an environment of the vascular region.

33. The method of claim 30, further comprising:
interconnecting a plurality of said packages;
moving said plurality of packages into the vascular region; and
filling the vascular region with a selected geometry of said plurality of packages.

34. The method of claim 33 wherein said plurality of said packages are operable to be positioned in a selected geometry substantially freely.

35. The method of claim 30 wherein guiding said package includes passing said package through a catheter.

36. The method of claim 35 wherein passing said package through said catheter includes moving said package with a hydrological force.

37. An embolization system comprising:
a flexible conduit;
a plurality of spherical functional units disposed at spaced intervals along said flexible conduit; and
a bioabsorbable polymer carried by each of said plurality of spherical functional units.

38. The embolization system of claim 37 wherein said bioabsorbable polymer is a polyglycolic acid.

39. The embolization system of claim 37 wherein said bioabsorbable polymer is a polylactic acid.
